(19) United States
(12) Patent Application Publication
(10) Pub. No.: US 2011/0177316 A1
(43) Pub. Date: Jul. 21, 2011

(54) LYZABLE MOLDED PARTS

(75) Inventors: Klaus Rennebeck, Wendlingen (DE); Bernd Hildenbrand, Vogtsburg (DE)

(73) Assignee: LEONARDIS-STIFTUNG, Stuttgart (DE)

(21) Appl. No.: 13/122,176

(22) PCT Filed: Oct. 3, 2008
(86) PCT No.: PCT/DE2008/001634

§ 371 Date: Apr. 1, 2011

(51) Int. Cl. B29C 71/00; B29C 39/00; B29C 47/00; B29C 49/00; B29C 51/10; B05D 1/08; B29C 35/08; C07K 14/415; C07K 14/78; C07C 59/265; C07C 55/10; C07C 229/24; C07H 19/207; C07H 19/20; C09F 1/00; C09K 3/00; D02G 3/00; B82Y 30/00

(52) U.S. Cl. 428/220; 264/234; 264/209.1; 264/210.1; 264/544; 264/571; 427/446; 264/494; 264/488; 530/356; 530/354; 562/584; 562/590; 562/571; 536/26.24; 536/26.26; 536/26.21; 536/26.25; 530/201; 530/200; 252/182.11; 428/401; 977/773

(57) ABSTRACT

The invention relates to a method for producing a biodegradable molded part, comprising: providing a binding agent which contains a lyzable biopolymer; forming a molded part from the binding agent, wherein the time stability of the molded part to biological lysis is set by at least one of the following measures: adding a pulverized inorganic solid matter in a proportion of 5% to 85% of the mass of the binding agent before forming the molded part; thawing at least one of the surfaces of the molded part using a chemical or biological method such that the surface has a structuring in the range between 1 nm and 10 μm. The invention further relates to an accordingly produced molded part.

The invention relates to a method for producing biodegradable molded parts like films and fibers, as well as to such molded parts.

The expression “biodegradable” is to be understood within the frame of the present invention as comprising each degradation process by means of micro-organism or enzymes thereof, according to which a molecular structure present in the molded parts is split up. An example of such a biodegradation process is the (human or animal) digestion. Especially, biological lysis falls within that expression.

Polymers have experienced a rapid propagation in industrial products of various kinds since their first chemical synthesis. The development of new polymers has resulted in versatile plastics with a great variety of properties such as temperature stability, moldability, heat-conductivity and so on.

In recent times, the focus of new developments was put increasingly on so-called “bio-polymers”, which mean, on the one hand, polymers synthesized from renewable raw materials, and, on the other hand, biodegradable plastics. Between both kinds of bio-polymers, there exists an intersection, which is constituted by polymers on the basis of naturally renewable raw materials which are also biodegradable. All those kinds of bio-polymers are presently the object of intense researches in order to provide ecologically compatible raw materials for industrial products. Examples for the use of such materials are especially packaging films or fibers for use in the production of textiles.

The expression “bio-polymer” as it is used for describing embodiments of the invention below, is based on the first one of the mentioned definitions and thus describes a polymer synthesized by renewable raw materials.

For different uses of bio-polymers, distinct life times (durabilities) are desired, which means that the requirements as to the resistance such a polymer brings forward against its micro-bacterial or enzymatic degradation, differ according to the kind and the use of a molded product produced therefrom.

It is an object of the present invention to satisfy those requirements and provide a method of producing biodegradable molded parts with which the life time (i.e. term of stability i.e. durability) of such molded parts can be set to a desired range. Furthermore, it is an object of the present invention to provide a corresponding molded part.

The object is achieved by a method according to claim 1 as well as a polymer molded part according to claim 13. Embodiments are given in the sub-claims.

A method for manufacturing a biodegradable molded part comprises:
providing a binding agent containing a lyzable bio-polymer;
forming a molded part from the binding agent;
wherein temporal stability of the molded part against biological lysis is set by at least one of the following measures:
a) adding a pulverized inorganic solid in a proportion between 5% and 85% of the mass of the binding agent before formation of the molded part;
b) thawing at least one of the surfaces of the molded parts by means of a chemical or biological process so that the surface comprises a structuring or pattern in a range between 1 nm and 10 μm.

Thus, the invention is directed to the control of the biological degradability in terms of time dimension. Products manufactured by a method according to embodiments of the present invention can therefore be adapted according to specific demands of the desired use in terms of their durability and lifetime, respectively.

According to one embodiment, the method aims at directed patterning/structuring the surface of a molded part which contains a lyzable bio-polymer, in order to set the desired degradability in view of a length of time of the degradation process. In this connection, the so-called “lotus effect” is applied, which is used by the present method for the purpose of controlled biodegradation. The expression “lotus effect” describes the fact that a surface patterned in a region of micrometers and nanometers, respectively, shows a poor wettability. Water as well as other liquids which deposit on the surface, do not adhere thereto or adhere only to a limited degree, but roll-off instead.

The lotus effect can, on the one hand, be achieved by adding a pulverized solid, especially an inorganic solid, to the binding agent. The particle size of the pulverized solid is approximately in the range of few nanometers to several micrometers. Especially, the particle size can be in the range between about 1 nm and about 10 μm. In this connection, the expression “particle size” means the average particle size of the pulverized solid, as for example pulverized bone marrow, hydroxylapatite (HA), diatoms, feather dust (pulverized bird feathers), kieselguhr (diatomaceous earth) and/or alumna. If the particles are present as nanoparticles, the average particle size is below 1 μm, especially preferred below 100 nm. The microparticles or nanoparticles in the binding agent cause a structuring of the surface of the molded part to be produced so that the lotus effect described above is achieved. As a rule, the density of the molded part is the higher, the smaller the pulverized solid particles contained therein are. A higher density has the effect that the lyzability of the molded part decreases, which means that the time period up to complete decomposition of the molded part increases. This effect arises in addition to the lotus effect already mentioned.

Preferably, the powders (pulverized solids) for admixing to the binding agent are present as high-purity powders. The pulverized solid is therefore preferably nearly free from impurities (purity>99.9%) and foreign matter, respectively.

A further possibility for the directed induction of the lotus effect in a molded part is the patterning/structuring of a finished surface of a molded part by means of a physical or chemical treatment. Among the plurality of surface treatment processes, such processes are suitable, which allow achievement of a pattern/structuring on the surface of the binding agent, which has the desired fine surface roughness. In this connection, for example, treatment of the surface by means of ultraviolet radiation or isotope radiation as physical surface treatment processes can be taken into account. Also pyrolytic coating of the surfaces of the molded part is a suitable process for applying surface patterns in the range of micrometers or nanometers.

Pyrolytic coating generally takes place in a temperature range of between about 500° C. and about 900° C. The hot pulverized material comprising particles a range of several nanometers up to several micrometers, can, for example, be spray-coated on the molded part, which is also heated.

In contrast to the surface treatment processes of irradiation with ultraviolet radiation or ion radiation mentioned above, the pyrolytic coating is a process with which the existing surface is not thawed, but a further surface layer is applied onto the existing surface. In addition to the pyrolytic coating, of course other suitable coating methods can be applied.

However, treatment by pyrolysis can also be applied as an isolated process (i.e. not in connection with coating) for patterning a surface. In this method, recesses are burned into the molded part by means of heating. In connection with thin films or hollow fibers, also pyrolyzing through the film or hollow fiber is possible, i.e. the generation of holes instead of mere recesses.

Finally, also surface treatment using tannic acids, aldehydes, salts, alcohols and oils as well as talc, waxes and carbon blacks can be carried out.

When irradiating the molded part with ultraviolet radiation, intensity and duration of the treatment depend from a composition of the binding agent as well as from the desired pattern/structuring. Generally, however, it can be mentioned that for structuring a molded part for the formation of a surface having a desired degree of roughness, irradiation of the molded part with ultraviolet rays having a frequency about 250 to about 350 nm, especially about 280 to 320 nm, for a time period from about 30 s to about 30 min is necessary.

As already mentioned, instead of irradiation with ultraviolet radiation or in addition to this, also irradiation with ion beams for structuring the surface of the molded part can be used. As an ion source radon can be used, for example. The duration of the irradiation can, for example, be chosen between several seconds and about 30 min. Especially, a duration of about 20 s to about 10 min is suitable for the formation of a patterned surface.

Besides the mentioned measures for achieving a lotus effect, the stability versus degradation like lysis or digesting can also be influenced by the composition of the binding agent. Especially, a proportion of products from the so-called “citric acid cycle” as well as from the cell respiration cycle in the binding agent is an important factor in adjusting the lyzability. This comprises, for example, the compounds NADH, ATP (adenosine triphosphate), ADP, succinic acid, aspartic acid, FAD, citric acid, butyric acid, and lactic acid. The better the proportions of those materials match, the more directed the biological decomposition takes place.

Further binder components which can influence the degree of the hydrophobic properties and hydrophilic properties, respectively, and/or the degree of lypophobic and lypophilic properties, respectively, are: silicon compounds, especially soap stone and talc, minerals, silicone oil, lanolin (wool fat), uric acid, oxalic acid, bone china, feather powder, tartaric acid, shellac, albumen, kieselguhr, alumna, cysteine, resilin.

Furthermore, the composition of the binding agent also influences the dioxygen permeability of the molded part. If the binding agent does not contain starch, the molded part is essentially impermeable for dioxygen. If there are amino acids in the binding agent, those amino acids also contribute to dioxygen impermeability. If the binding agent contains a starch polymer and an amino acid polymer, which are not in a homogeneous mixture, the resulting molded part is selectively permeable. Depending on the locations within the molded part at which starch containing areas are located, the molded part is dioxygen permeable at those locations, whereas at those locations, which contain amino acids (for example collagen), it is dioxygen impermeable.

The molded part can be formed so that it is fully or partly lyzable. For example, it is possible to form a surface by means of the mentioned structuring using the mentioned processes so that wettability is reduced in comparison to a smooth surface. Therefore, water, working fluids or other fluids which may be completely free from water, where appropriate, can affect those surface to a lesser degree, and therefore the decomposition process of the biodegradable molded part is decelerated from the respective side. The process of dissolving/decomposition of the biodegradable molded part progresses in an asymmetric way in this case, with a stronger progress from the side having a smooth surface and a reduced progress from the structured side.

As to suitable materials for the binding agent, the binding agent can contain one or several of the following components: bio-polymers like blood, components of blood, proteins, peptides, cellulose, starch, cutin, acetate, glycerol, alcoholates, resins, waxes, agar, collagens, tallow, fats, cysteine, gelatine, bone glue, neatsfoot oil, fish oil, gristle glue, skin glue, animal glue, fish glue, urea, casein, polymers, especially polyvinyl alcohol, polyethylene, polypropylene, polyvinyl ester, polyvinyl acetate, polyamines, polyacrylics, polyester, polyamide, polyimide, polysulfone, polysulfide, polystyrene, copolymers of cellulose. In this connection, the binding agent can be added as a solid, if it is fusable, or in powder form, if it is homogenized afterwards with a suitable fusable binding agent, especially preferred with a resin, wax, especially wool fat (lanolin), linseed oil, hempseed oil, boiled linseed oil, terpene, sole-gel, fresh egg, sodium silicate, CS₂, carbon sulphide, methyl chloride, methyl, silicone oil, urea, polyvinyl alcohol and/or a suitable solvent. Besides, the binding agent may contain molten solids containing anions or cations (positive ions) for the molding process, depending on the processing method.

If the binding agent is to possess elastic properties and, at the same time, be biodegradable, then resilin is suitable as a component for the binding agent.

Especially, the binding agent for carrying out the method should be in a sufficiently liquid state, i.e. pourable, sprayable, extrudable or spinnable. If the lyzability is set by means of a pulverized solid, then the pulverized solid is admixed to the binding agent before molding or is applied by means of a coating process, for example, by means of pyrolytic coating. Preferably, the binding agent has a gelling ability of about 160 bloom before processing.

In connection with one of the previously mentioned binding agents, also a sole-gel can be used, for example in the form of saline compounds, with which nanoparticles are mixed or which contains nanoparticles as such.

The pulverized solid preferably contains at least one of the following components: oxides, silica, zeolites, silicon, silicon compounds, burned-up bone ash, hydroxyl apatite, metals, silane, magnetite, hematite, iron pentacarbonyl, lithium chloride, anatase, rutil, zinc chloride, lithium, zinc, manganese, selenium, rare earths, perovskites. If again fusible/meltable binding agents are used, the pulverized solid preferably contains SiO₂, TiO₂, ZrO₂ and/or Al₂O₃. Generally, the pulverized solids may be either organic or inorganic. Inorganic solids can also be ion conductors as for example zirconium oxides. Besides, also biological proton conductors can be used, which are sulfonated, fluorinated, phosphorylated or constitute a combination of those substances.

A molding step can comprise, for example, spraying, casting, extruding or spinning. In this manner, the lyzable molded part can be formed as a film, fiber or hollow fiber. The molded parts can be produced in a multilayer form or in a single-layer form. A multilayer form, especially a two-layer form, is especially applicable, if an asymmetric lyzability of the molded part is desired. Methods for the production of such double-layer or multilayer molded parts are, for example, disclosed in DE 10 2005 056 491. The methods of spinning, extruding, casting or spraying for multilayer molded parts described therein are applicable to the embodiments of the present invention.

If a double-layer or multilayer form is desired, the respective binding agent masses can be treated either in one process step or in two or several successive process steps. The expression “treatment” is to be understood here as a step of molding a molded part. If two or several process steps are chosen, those process steps are carried out, according to one embodiment of the invention, one after the other as to their timing. Care is to be taken that a time sequence of the process steps is sufficiently rapid, so that the mass treated first does not form a skin layer or only forms a minimal skin layer on its surface. In this case, there is no casting or coating.

A lyzable or partly lyzable molded part according to one embodiment of the invention comprises a binding agent, which contains a lyzable bio-polymer, the binding agent containing at least one of the following components: herbal collagens, animal collagens, gelatine, acids of the citron acid cycle, reaction products of the cellular respiration chain, resilin. The proportion of products from the citric acid cycle as well as the cellular respiration chain in the binder is of importance for setting the lyzability. This comprises the compounds NADH, ATP, ADP, succinic acid, aspartic acid, FAD, citric acid, varnish as for example shellac (lacca in tabulis). The more differentiated the proportion of those components is in the binding agent, the more directed is the biodegradation.

In this manner, on the one hand the biodegradability and/or chemical degradability of the molded part is assured. In this manner, it is ensured that the molded part has a finite lifetime. On the other hand, the molded part has properties which may decrease the degradability or lyzability so that the time period for degradation is prolonged. This constitutes a retardation of the biological or chemical lysis.

At least one of the surfaces of the molded part can comprise a structuring/patterning in the range of micrometers or nanometers. As already described, this structuring can be achieved by treatments like photo-chemical patterning, irradiation with ultraviolet radiation or ion beams, by adding of microparticles or nanoparticles to the binding agent mass of the molded part to be formed. Besides, there exists the possibility that the molded part is a fabric or tissue, which is knitted, woven or otherwise formed. The molded part can also be an accumulation of bulk fibers which are kept together by means of a binding agent, for example by means of an adhesive.

The expressions “micro hollow fibers” and “nano hollow fibers”, respectively, are meant to designate within the context of the embodiments of the present invention, fibers the equivalent outer diameter of which lies between one or more micrometers and nanometers, respectively. Such fibers can comprise wall thicknesses between about 8 nm and 800 nm, especially between 30 nm and 380 nm, and, according to some embodiments, between 50 nm and 180 nm. The lengths of the fibers can be approximately 30 mm to approximately 300 mm. Of course, also longer or shorter fibers can be produced according to the requirements and can be used in the context of the present invention. Thus, corresponding textile staple fibers can be produced up to a length of about 20,000 m. The micro or nano hollow fibers which are further processed to a woven fabric or knitted fabric, can have an even wall thicknesses over the length of the fibers.

Such fine hollow fibers can be produced by spinning of the binding agent which contains the degradable and lyzable, respectively, bio-polymer. In order to spin such fine structures, use of an atomic force microscope is required. Such microscopes are known in the state of the art, and therefore, no detailed description of their operation will be made. In the interior of the spinning nozzle, which is used for the spinning of the fine hollow fibers, there is positioned a lumen maker. This lumen maker is made from a metal or a metal alloy. Especially, materials like tantalum, tungsten, titan or alloys which contain those metals, are suitable as starting material for the lumen maker. The lumen maker can then be inserted into a spinning nozzle of ceramics or any other suitable material, for example. For producing hollow fibers in the range of micrometers, the lumen maker has a diameter of, for example, 6 nm to 20 μm. The biologically lyzable hollow fibers spun out of such a spinning nozzle already show their final contours. A stretching of the fiber after the spinning process is usually not necessary.

As already mentioned, besides spinning, also extrusion is a suitable molding process in order to produce a molded part according to an embodiment of the invention. As a starting material for an extrusion process (as well as for the other molding processes mentioned), the binding agent can be provided as a molten mass/melt. Subsequently to the molding process, for example the extrusion process, the molded part can be solidified and cooled by means of a cooling agent in the form of a water-free liquid that becomes lost. In this connection, the expression “getting lost” means that the liquid permeates in the course of the cooling process into the wall of the molded part to a certain depth, mixes with the binding agent and thus gets a component of the wall of the molded part. Therefore, it constitutes a so-called reaction cooling agent. According to one embodiment, the cooling agent may also comprise slippage enhancing properties. As an example for such a reaction cooling agent comprising slipping lubrification, silicone oil is to be mentioned. The silicone oil is acting in a cohesive manner, however, without affecting the lyzability. Furthermore, the rigidity of the wall into which the lubrificating cooling agent is incorporated during the processing, is enhanced.

By means of the cooling step after the molding process, also structuring of the surface of the molded part can take place. As a rule, the faster the cooling-down takes place, the more distinct the structural pattern of the surface will become, which means the deeper the structure will be formed into the surface. The structuring, which is in a depth range of several nanometers, can, for example, be made using a transmission microscope.

In order to extrude a binding agent (which contains nanoparticles, where appropriate) from the previously mentioned materials, cooling may be applied during the extrusion process. This cooling may be effected, for example, in the form of a cooling bath downstream of the extruder. Furthermore, a cooling fan can additionally or alternatively be provided around the extruder, or there can be one or more cooling channels which are formed in the cylinder. In this manner, also hydrolysis products of animal cell tissues from poultry, game animals, fish or other domesticated animals or farm animals can undergo the molding process. The technically more complex and more expensive injection molding can therefore be avoided.

With the methods mentioned above, it is possible to carry out endless extrusion or endless spinning, i.e. a molding without interruption of the extrusion process or spin process. This means that fibers of any length can be produced. Those fibers can then undergo a further treatment by common fiber processing methods. In this manner, also woven fabrics or knitted fabrics can be produced from the lyzable fibers. Both molding processes have in common the formation of a hydraulically equivalent diameter of the hollow profile of the fibers.

If for the structuring of one or more of the surfaces of the molded part, nanoparticles in the form of a solid are used, there is achieved the additional advantage that the binding agent is provided with additional rigidity and stability. This effect is especially advantageous in connection with molded parts of a low wall thickness, because the dimensional stability of those molded parts can be enhanced in this manner in comparison to molded parts having the same binding agent, but not containing solid nanoparticles. Furthermore, the pulverized solid can also comprise the properties of an electrolyte within the molded part, if a suitable material for the solid is chosen. In this connection, for example, titanate can be used.

According to one embodiment of the invention, the binding agent can also contain silicon or silicon compounds. Therefore, according to one embodiment silanes (hydrosilicons) (mono-silanes or long-chain silanes) can be introduced into the bio-polymer binding agent in a sealed manner. Afterwards, the molded part is formed in of the manners described above. By means of heating in connection with molding, the silanes react with oxygen of the air and produce pure silicon and silicic acid. This silicic acid in turn influences the lyzability of the produced molded part in such a manner that the lyzability related to water is adjustable with an increasing proportion of silicic acid. Further possible reaction products of the decomposition of the silanes are hydrogen and kieselguhr which accelerate the degradation and lysis, respectively, of the finished molded part.

In order to enhance the elasticity of the finished molded part, the binding agent may contain, for example, resilin and/or collagens. The elastic properties of a molded part produced from the mentioned materials are enhanced, if the binding agent is added glycerol, sugar and/or starch.

Hollow fibers which are produced from binding agents containing collagens, can, depending from the proportion of collagen in the binding agent, be impermeable for oxygen, but permeable for nitrogen. For this reason, they can be used for the separation of air and for the separation of the air components, respectively. The fibers can be used in a sintered state as well as in an not sintered state. A similar effect can also be obtained if a hollow fiber made from lyzable bio-polymers contains solid nanoparticles of zirconium oxides. The zirconium oxide in the binding agent has the effect that oxygen ions are conducted through the walls of the molded part. The sintered fibers containing a proportion of ceramics or metal can then be used as catalytic converters, for photo-voltaic elements or as molecular sieve, recuperator and regenerator, as a thermogenerator as well as an exchanger for anions or cations.

The molded parts can be, for example, films having a thickness of one atom layer up to few atom layers up to 1 mm. For certain applications, it makes sense if the thickness of the film is between 1 μm and 300 μm. Moreover, the molded parts can be produced as hollow profiles, for example small tubes or hollow fibers. The hollow fibers can be textile fibers having an inner diameter of 80 nm to 30 μm. The wall thickness of the hollow fiber can, for example, correspond to the film thickness. Of course, the molded parts can also be full fibers and small rods without an inner hollow space (lumen).

The molded parts according to the embodiments of the invention can be used for a variety of purposes. The fact that a molded part can be formed from a lyzable bio-polymer used as a binding agent, the lyzability of the molded part being adaptable to a respective use, makes it possible to produce numerous products for one-way use from biodegradable materials.

A potential field of employment of such molded parts are disposable cosmetic products or toilet requisites. In this connection, the lyzability is to be set so that the molded parts are stable until use, but dissolve or degrade in sewage water within a time period as short as possible, i.e. within 10 min to about one week. One embodiment of the invention is therefore directed to a swab (ear swab), the rod body of which is produced from a lyzable bio-polymer binding agent, the surface of the rod body being structured so that lysis does not take place immediately after contact with water.

A further embodiment is directed to a textile fabric or, more generally, a textile structure made from textile fibers or hollow fibers, which are formed from a binding agent comprising a lyzable bio-polymer. The lyzable bio-polymer can comprise components of natural collagens as well as residues of process steps of different hydrolysis reactions. Those oxidation residues can constitute, in form of ashes, the pulverized solid, which is introduced into the binding agent for affecting the lyzability.

The textile structures and textile fabrics, respectively, can be a cosmetic tissue or a diaper for children or in case of adult incontinence, according to embodiments of the invention. Especially in the case of the diaper, it is important that the binding agent comprises a certain degree of elasticity, which is the reason why resilins or collagens, as already mentioned, can be contained.

According to other embodiments, also use as technical textiles is possible. These are especially redox batteries or lithium ion batteries as well as exchangers for anions and cations, recuperators and regenerators. The fibers and hollow fibers, respectively, from which such textiles are made, are preferably baked and sintered, respectively, after spinning or extrusion.

If the surfaces of the molded part are formed impermeable for oxygen or only selectively, i.e. partially, permeable for oxygen by use of collagens and under omission of starch polymers, the molded part can also be used as an anti-corrosive means, for example for auto vehicles in the form of an underbody protection. For this, the molded part is formed as a film from a bio-polymer as well as from silica in a proportion of 40-70%, especially about 50-65% silica. The advantage of an anti-corrosive made from such a collagen containing molded part lies in the fact that it acts, inter alia, in a fume reducing manner, which is especially favourable in connection with motor vehicles. Moreover, a water-free liquid can be used as a cooling agent or as a combined cooling/lubrification agent for the molding process, for example the extrusion process, as already mentioned. The cooling agent and the cooling/lubrification agent (for example silicone oil), respectively, is added to the melt of the binding agent prior to filling a screw extruder, however, without mixing both components. Afterwards, the molding process and the subsequent cooling of the extruded molten mass in an immersion bath, for example at room temperature, take place. The cooling agent or cooling/lubrification agent can also be constituted by the fill of the immersion bath.

As an alternative molding method, also vacuum pressure molding can be used. In this way, films are produced which can be baked or sintered afterwards, as required.

With reference to the extrusion process, it is also possible to produce granulates on the basis of the lyzable bio-polymers.

In the following, embodiments of the invention are described with reference to the drawings, wherein the embodiments are to be understood as non-limiting examples. In the drawings:

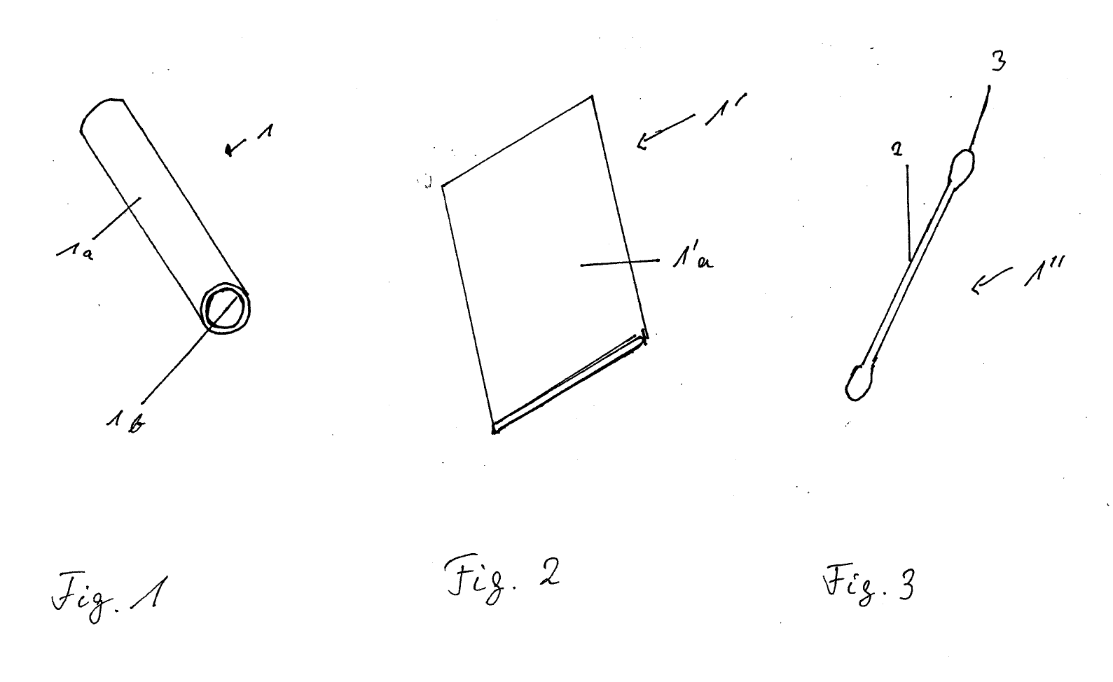

FIG. 1 shows a first molded part according to one embodiment of the invention, which is formed as a hollow fiber;

FIG. 2 shows a second molded part according to another embodiment of the invention, which is formed as a film;

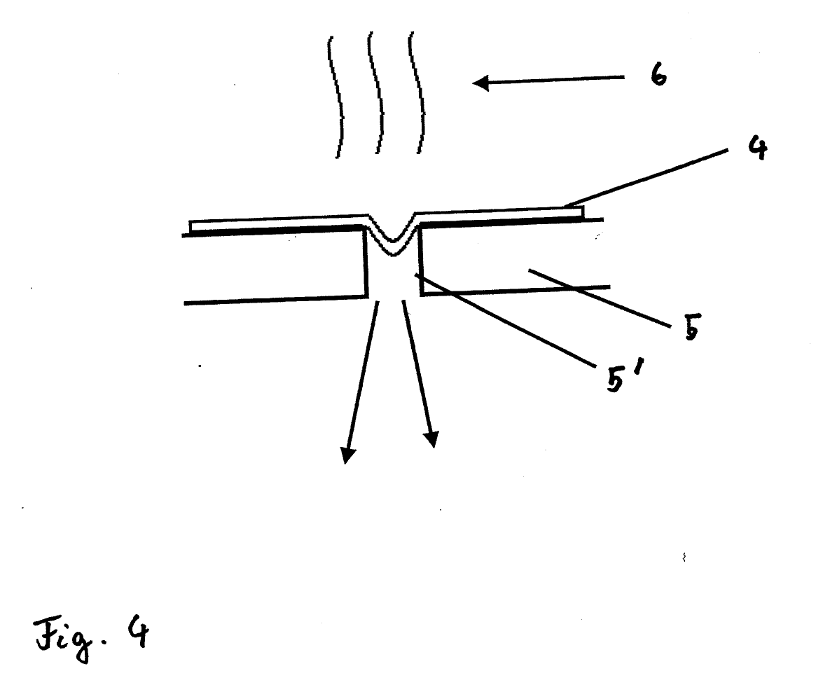

FIG. 3 shows a third molded part according to a further embodiment of the invention, which is formed as a swab; and

FIG. 4 shows a method according to one embodiment of the invention according to which the molded part is formed as a deep-drawn/thermo-formed fiber.

In FIG. 1, there is shown a first embodiment of the invention. This embodiment constitutes a molded part 1, which is formed as a hollow fiber here. The hollow fiber may be, for example, a micro-hollow fiber, which means that its outer diameter is smaller than 1 mm.

The hollow fiber is formed here with a single layer, i.e. the complete wall is essentially homogenous as to its composition. Of course, it is also possible to form a double-layer or multilayer hollow fiber, so that in a thickness direction of the walls, there are present layers of different compositions. With other words, the hollow fiber is inhomogenous in the thickness direction of the walls.

The hollow fiber comprises an outer surface 1a, as well as an inner surface 1b. One or both of the surfaces 1a, 1b can comprise a patterned structure so that the lyzability of the binding agent, which is the basis compound of the hollow fiber, is restricted. With other words, the time period up to the gradation of the molded part 1 is decelerated at the respective side of the wall. The hollow fiber can be formed by an extrusion process or a spinning process.

The hollow fiber shown in FIG. 1 can be further processed to form textile structures, for example woven fabrics or knitted fabrics. Such textile structures can be employed especially as a diaper or cosmetic tissues.

FIG. 2 shows a further embodiment of the invention. The molded part 1′ of this embodiment is a film, which is also shown as a mono-layer film here. Due to the fact that the film does not constitute a hollow profile, in contrast to the hollow fiber of the first embodiment, this embodiment has only one outer surface 1′a. Also this surface can comprise a structuring/pattern.

The embodiment of FIG. 2 can be further processed in various ways, especially to form a deep-drawn (thermo-formed) cylinder, a small tube, catheter or hollow fiber. For this, the deep-drawing (thermo-forming) process already mentioned can be employed, which will be explained in more detail with reference to FIG. 4 further below. The deep-drawing process is a vacuum thermo-forming process according to which, in a first step, a tube or small tube closed at one end or a closed fiber can be produced, which can then be opened in a further process step (for example by cutting).

Production of the lyzable film as shown in FIG. 2 is possible, for example, by means of an extrusion process.

FIG. 3 finally shows a third embodiment of the invention. In this case, the molded body 1″ is formed as a swab, which resembles a usual cotton swab in terms of its contour.

In the embodiment shown, the swab consists of a shaft body 2 as well as two bulgy ends 3 which serve for cleaning. The shaft body 2 and the ends 3 may be produced from the same or different materials. The shaft body 2 can be molded in an extrusion process out of a screw extruder. The bulgy ends are deposited afterwards in a separate process step.

The swab, which is formed from a binding agent of the previously mentioned materials, may be adapted as to its lyzability, so that it is completely degraded within sewage water after about 10 minutes up to about one week.

As an example of a composition of a binding agent for a swab, according to which the shaft body 2 and the ends 3 are made from the same materials, the following values may be given: 50% zirconium oxide and 50% gelatine or 60% zirconium oxide and 40% gelatine or 60% silica and 40% gelatine. The binding agent can be provided as a granulate material or crashed material for melting, for example, in an extruder.

The melt/molten material is then processed to form a little rod, which resembles the shaft body 2 of the swab. The thickness of the little rod or little tube can be about 3 mm to 6 mm, especially about 4 mm to 5 mm, according to the requirements. After the extrusion process, the little rod or little tube is cooled in an immersion bath which consists preferably of a completely water-free liquid cooling/lubrification agent, for example silicone oil. The cooling/lubrification agent in the immersion bath is tempered to about 30° C. to 60° C., so that cooling and solidification of the hot extruded small rods and small tubes, respectively, takes place. Due to the lubrification property of the cooling liquid, conglutination (gluing) of the extruded small rods is avoided. Besides, the cooling/lubrification agent has also the effect that emission of odors of the binding agent can be avoided.

The ends 3 of the swab are formed from the same materials as the shaft body 2, i.e. from the materials mentioned above. In contrast to the production of the small rods, the binder material is provided here as textile dust particles, which may be a by-product in the production of the small rods. The dust particles can be fluffed-up as to their volume by means of pressurized air. Subsequently, the wad (wadding) produced in such a manner, is mounted to the shaft body, if applicable under use of an adhesion enhancing agent, as for example a small amount of water.

In FIG. 4, there is shown an exemplary production process for a fiber or a small tube or a small rod according to one embodiment of the invention. A binding agent, which contains a lyzable bio-polymer as well as a pulverized solid, if required, is deposited as a film 4 onto a perforated block 5. Then, heating of the film 4 takes place, for example by means of infrared radiation, which is indicated here by means of sinous lines 6. The film is heated until it is pourable and drawable, respectively.

The perforated block 5 comprises openings 5′, which extend through its whole thickness. In FIG. 4, only one opening 5′ is shown for sake of simplicity of the illustration, but it is obvious for the person skilled in the art that more openings 5′ may be present, which extend in parallel through the thickness of the block 5. At the lower side of the perforated block 5 opposite to the film 4, a low pressure is produced so that the pourable heated binding agent of the film 4 is drawn through the opening as indicated by arrows. At the lower end of the opening 5′, a small tube emerges, which can be cooled and solidified in the manner already described.

A molded part produced by a method according to an embodiment of the invention can be lysed in use starting from one of its surfaces or from several of its surfaces. If the molded part is, for example, a hollow profile, then, in one case, the lysis can take place starting form the outside towards the inside or vice versa. In another case, the lysis may progress uniformly from the inner and outer surfaces of the hollow profile. Finally, also a case is possible according to which the lysis starts on both surfaces, but progresses with different velocities from the outer surface and the inner surface.

The asymmetric progress of the lysis of a molded part according to one embodiment of the invention can either be effected in a directed manner by forming the different surfaces with different materials for the binding agents and the pulverized solid contained therein, respectively, by forming the different surfaces with different grains sizes of the solid and/or with different surface structurings/patterns. The expression “different surface structurings” can mean, in this connection, that the structuring of the surfaces differ from each other in terms of their depths (i.e. in terms of the degree of roughness), in terms of the grain densities and/or in terms of the grain sizes. In this manner, a lotus effect of different degrees provides for the mentioned asymmetry. Besides, the different progressions of the lysis may also be due to the environmental conditions to which the different surfaces are exposed.

It is to be mentioned that the embodiments of the invention given above are to be understood as non-limiting examples only.

1. A method for producing a biodegradable molded part, comprising:
providing a binding agent which contains a lyzable biopolymer;
forming a molded part from the binding agent;
wherein temporal stability of the molded part against biological lysis is set by at least one of the following measures:
a) adding a pulverized inorganic solid in a proportion of 5% to 85% of the mass of the binding agent before forming the molded part;
b) thawing at least one surface of the molded part using a chemical or biological method such that the surface has a structuring in a range between 1 nm and 10 μm.

2. The method of claim 1, wherein the pulverized inorganic solid comprises nanoparticles.

3. The method of claim 1, wherein the pulverized solid contains at least one of the following components: oxides, silicic acid, zeolites, silicon, silicon compounds, burned up bone ashes, hydroxyl apatite, metals, silanes, magnetite, hematite, iron pentacarbonyl, lithium chloride, anatase, rutil, zinc chloride, lithium, zinc, manganese, selenium.

4. The method of claim 3, wherein the pulverized solid comprises one or more oxides out of the following group: vanadium oxide, titanium oxide, tungsten oxide, cobalt oxide, ferric oxide.

5. The method of claim 1, wherein the binding agent comprises at least one of the following components: herbal collagens, animal collagens, gelatine, resilin.

6. The method of claim 1, wherein the binding agent comprises acids of the citric acid cycle and/or reaction products of the cellular respiration chain.

7. The method of claim 1, wherein the molded part is formed as a film or a fiber, especially as a hollow fiber.

8. The method of claim 7, wherein formation of the molded part is carried out according to one of the following methods: film casting, film blowing, film extrusion, extrusion of a hollow fiber, deep-drawing of a film, wraping of a hollow fiber from a film, deep-drawing of a fiber from a film.

9. The method of claim 8, wherein thawing of the at least one surface is carried out by means of coating a foil with a cover layer containing nanoparticles.

10. The method of claim 9, wherein coating of the molded part is performed by means of pyrolysis.

11. The method of claim 1, wherein the thawing of the at least one surface of the molded part is carried out by irradiating the molded part with ultraviolet radiation of a frequency between 250 nm and 350 nm over a time period between 30 s and 30 min.

12. The method of claim 1, wherein the thawing of the at least one surface of the molded part is carried out by irradiating the molded part with isotope radiation over a time period between 5 s and 20 min.

13. A lysable molded part comprising a binding agent which contains a lysable biopolymer, wherein the binding agent contains at least one of the following components: herbal collagens, animal collagens, gelatine, acids of the citric acid cycle, reaction products of the cellular respiration chain, resilin, wherein at least one of the surfaces of the molded part has a structuring in a range of micrometers or nanometers.

14. The lysable molded part of claim 13, wherein the molded part comprises an inorganic solid in a proportion between 5% and 85% of the mass of the binding agent, the inorganic solid being present in the form of nanoparticles.

15. The lysable molded part of claim 13, wherein the molded part is formed as a hollow fiber having an inner diameter between 80 nm and 30 μm.

16. The lysable molded part of claim 13, wherein the molded part is formed as a film having a thickness between one atom layer and 1 mm.

17. The lysable molded part of claim 13, wherein the molded part is formed as a swab.

18. The lysable molded part of claim 13, wherein the molded part is formed as a fibrous web.

19. The lysable molded part of claim 13, wherein at least two surfaces of the molded part differ from each other in terms of their structurings.

20. The lysable molded part of claim 13, wherein at least two surfaces of the molded part differ from each other in terms of composition of the binding agent.
